(19) United States
(12) Patent Application Publication
(10) Pub. No.: US 2022/0168258 A1
(43) Pub. Date: Jun. 2, 2022

(54) METHOD FOR TREATING FEMALE NON-SMOKERS WITH NON-SMALL CELL LUNG CANCER

(71) Applicant: Lantern Pharma Inc., Dallas, TX (US)

(72) Inventors: Aditya Kulkarni, Dallas, TX (US); Arun Asaithambi, Dallas, TX (US)

(21) Appl. No.: 17/469,765

(22) Filed: Sep. 8, 2021

Related U.S. Application Data
(63) Continuation of application No. PCT/US2020/021615, filed on Sep. 17, 2020.
(60) Provisional application No. 62/815,762, filed on Mar. 8, 2019.

(51) Int. Cl. A61K 31/255; A61P 35/00; A61K 45/06

(52) U.S. Cl. CPC A61K 31/255; A61K 45/06; A61P 35/00

(57) ABSTRACT

A method of treating a female patient suffering from non-small cell lung cancer includes the step of administering to the patient in need thereof a composition of 2,2′-dithio-bis-ethane sulfonate, or a pharmaceutically-acceptable salt thereof. The method can include other primary line therapies.

### TECHNICAL FIELD

This application relates to pharmaceutical compositions, methods, and kits used for the treatment of cancer and other medical conditions. More specifically, this application relates to pharmaceutical compositions, methods, and kits comprising medicaments used for the treatment of non-small cell lung cancer, advanced non-small-cell lung cancer, adenocarcinoma, and other medical conditions, particularly in females and nonsmoking or never smoking females.

### BACKGROUND

Tavocept or disodium 2′-dithio-bis-ethane (CAS No. 16208-51-8) is a small molecule (about 326 Da) that is water soluble, that can be delivered intravenously, and that has the following structure:

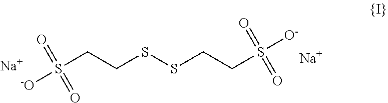

Tavocept demonstrated positive subgroup responses with significant improvements in overall survival but did not meet clinical efficacy endpoints.

[symbol]

Worldwide, lung cancer is the most common cancer in terms of both incidence and mortality. Lung cancer is the leading cause of cancer death worldwide among men and women combined and the cost of lung cancer care in the US in 2015 was $13.4 billion. The American Cancer Society estimates that of the 234,030 new cases of lung cancer in 2018 in the United States, 112,350 of those were in women. NSCLC is the most common form of lung cancer (app. 85% of lung cancers), with adenocarcinoma, squamous cell and large cell carcinoma as the three subtypes in decreasing prevalence order. Approximately 10-15% of all lung cancers arise in never smokers, making lung cancer in never smokers one of the leading causes of cancer-related mortality. Given the impact of this disease, there is surprisingly little information available on the descriptive epidemiology of lung cancer in never smokers. General population statistics are largely uninformative because neither cancer registries nor routinely collected death certificates provide reliable information on lifetime smoking histories. In addition, reports on smoking from next-of-kin or in medical records are incomplete and often unreliable. Only large-scale cohort studies can measure age-and sex-specific lung cancer rates in never smokers with reasonable precision, and these have generally studied mortality rather than incidence. Currently there is no approved therapy specifically for the growing indication of non-smokers with Non-Small Cell Lung Cancer or NSCLC.

Approximately 40% of all NSCLC are adenocarcinomas, while more than half are in women. The majority of people diagnosed with lung cancer today are not active smokers, and unlike the recent decrease in lung cancer in general, lung cancer is significantly increasing in one group of people: women who do not smoke. The prevalence of lung cancer in non-smokers has been increasing over time with over half occurring in current non-smokers.

Recent data suggest that lung cancer mortality rates among women are projected to rise globally by 43% by 2030 and exceed deaths from breast cancer. It has been argued that lung cancer in female non-smokers is a distinct type of cancer, but studies describing this population are scant. This population remains under-served and lung cancer in female non-smokers should be classified as a rare disease. The poor prognosis of advanced non-small cell lung cancer (NSCLC) is probably due tumor resistance to chemotherapy.

Accordingly, there is a need for a treatment for nonsmoking female NSCLC patients with adenocarcinoma. It is to this need, among others, that this application is directed.

### DESCRIPTION OF THE FIGURES

FIG. 1 shows retrospective subgroup analyses of NSCLC adenocarcinoma patients receiving Cisplatin and/or Paclitaxel;

FIG. 2 shows the percentage of patients undergoing treatment failure was the least among non-smokers receiving in the 2,2′-dithio-bis-ethane sulfonate; and

FIG. 3 shows the percentage of patients undergoing treatment failure was the least among females non-smokers receiving 2,2′-dithio-bis-ethane sulfonate.

### SUMMARY

This disclosure provides methods, devices, and compositions for distributing a combination of a cell division inhibitor (e.g., cisplatin, cisplatinum, or cis-diamminedichloroplatinum (II)) and a disodium 2′-dithio-bis-ethane to a non-small-cell lung cancer, adenocarcinoma patients.

One aspect of this application provides a combination therapy of disodium 2′-dithio-bis-ethane to treat non-small cell lung cancer, particularly female non-smokers. In some embodiments, the therapy is one or more chemotherapeutic agents selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, cetuximab and a Raf kinase inhibitor.

Another aspect includes a method of treating advanced and/or metastatic non-small cell lung cancer in female patients, the method comprising administering to a human patient having non-small cell lung cancer who has received a second-line or a higher-line therapy a pharmaceutical composition of 2,2′-dithio-bis-ethane sulfonate, or a pharmaceutically-acceptable salt thereof and a second therapeutic agent. The non-small cell lung cancer can be lung adenocarcinoma.

Another aspect includes a method for treating a female patient suffering from non-small cell lung cancer comprising the step of administering to the patient in need thereof a composition of 2,2′-dithio-bis-ethane sulfonate, or a pharmaceutically-acceptable salt thereof. The patient can a non-smoker or never smoker. The method can include the additional step of co-administering to the patient in need thereof a second therapeutic agent useful in the treatment of non-small cell lung cancer.

Another aspect includes a method of treating a female, nonsmoking patient suffering from, or susceptible to, non-small cell lung cancer comprising the step of determining whether the patient is a non-smoker; and administering to the non-smoker a composition of 2,2′-dithio-bis-ethane sulfonate, or a pharmaceutically-acceptable salt thereof. The method could include a second therapeutic agentis paclitaxel or cisplatin. Further the method could include the additional step of co-administering to the patient in need thereof a second therapeutic agent useful in the treatment of non-small cell lung cancer. The second therapeutic agent can selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, and cetuximab.

Another aspect includes a method that includes determining whether the non-small cell lung cancer is ALK, ROS, MET, EGFR mutant-positive non-small cell lung cancer. In instances, the non-small cell lung cancer includes ALK and ROS1 gene fusions/rearrangements, EGFR gene mutations/deletions, and MET/HGFR gene amplifications

The details of the invention are set forth in the drawing and description below. Other features, objects, and advantages of the invention will be apparent from the description and from the claims.

### DETAILED DESCRIPTION

One embodiment includes a method for increasing survival time in a female patient with non-small cell lung carcinoma or non-small cell lung carcinoma in which 2,2′-dithio-bis-ethane sulfonate or salt is administered in a therapeutically effective amount to the patient with non-small cell lung carcinoma. In one example, 2,2′-dithio-bis-ethane sulfonate or its salt may be administered either prior to, concomitantly with, or subsequent to the administration of a chemotherapeutic agent or agents. In one example, the female patients are non-smokers. In another particular embodiment, the method is used to treat a female, non-smoker patient suffering from to non-small cell lung cancer.

The compositions are a therapeutically-effective dose of an oxidative metabolism-affecting Formula (I) compound including, but not limited to, the disodium salt of 2,2′-dithio-bis-ethane sulfonate or a pharmaceutically-acceptable salt or analog thereof. The disodium salt of 2,2′-dithio-bis-ethane sulfonate has also been referred to in the literature as 2,2′-dithio-bis-ethane sulfonate. Various salts and analogs of 2,2′-dithio-bis-ethane sulfonate, as well as other dithioethers may also be synthesized as outlined in U.S. Pat. Nos. 5,808,160, 6,160,167 and 6,504,049, the disclosures of which are hereby incorporated by reference in their entirety. Additionally, the compositions of the present invention also comprise a medically-sufficient dose of the metabolite of disodium 2,2′-dithio-bis-ethane sulfonate, known as 2-mercapto ethane sulfonate sodium.

In another embodiment, any of the above methods of treatment comprises the further step of co-administering to the patient one or more second therapeutic agents. The choice of a combination of agent or second therapeutic agent may be made from any second therapeutic agent known to be useful for co-administration with 2,2′-dithio-bis-ethane sulfonate or salt. The choice of second therapeutic agent is also dependent upon the particular disease or condition to be treated. Examples of second therapeutic agents that may be employed in the methods of this application are those set forth above for use in combination compositions comprising a compound of this invention and a second therapeutic agent.

In another embodiment, the second therapeutic is one or more chemotherapeutic agents selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, cetuximab and a Raf kinase inhibitor.

In another embodiment, the second therapeutic is one or more chemotherapeutic agents selected from paclitaxel or cisplatinum.

Methods delineated herein also include those in which the patient is identified as in need of a particular stated treatment. Identifying a patient in need of such treatment can be in the judgment of a patient or a health care professional and can be subjective (e.g. opinion) or objective (e.g. measurable by a test or diagnostic method). 2,2′-dithio-bis-ethane sulfonate may target molecular pathways that are more common in female non-smokers than in any other group. c-Met, also called tyrosine-protein kinase Met/MET mesenchymal-epithelial transition/hepatocyte growth factor receptor (HGFR)/anaplastic lymphoma kinase (ALK), ROS-1 (orphan receptor tyrosine kisase) & epidermal growth factor receptor (EGFR) gene alterations are more common in non-smokers, who are most commonly female and present with advanced stage adenocarcinoma. In certain embodiments, patients may be first screened using one or more test for EGFR and c-Met/ALK status. A high percentage of adenocarcinoma patients appear to have either EGFR gene mutants or be c-Met/ALK positive or ROS-1. A method of treating advanced and/or metastatic non-small cell lung cancer in female patients, the method comprising: administering to a human patient having non-small cell lung cancer who has received a second-line or a higher-line therapy a pharmaceutical composition of 2,2′-dithio-bis-ethane sulfonate, or a pharmaceutically-acceptable salt thereof and a second therapeutic agent.

In one embodiment, an effective amount of a compound of this application can range from 10-40 grams per dose. In one embodiment, an effective amount of a compound of this application can range from 1-500 grams per dose. In some embodiments, an effective amount ranges from 0.01-10 grams per dose. In other embodiments, an effective amount ranges from 10-60 grams per dose. It is not necessary to provide an equal dosage per day or per week.

Therapeutically effective doses can vary, as recognized by those skilled in the art, depending on the diseases treated, the severity of the disease, the route of administration, the age and general health condition of the patient, excipient usage, the possibility of co-usage with other therapeutic treatments such as use of other agents and the judgment of the treating physician. For example, guidance for selecting an effective dose can be determined by reference to the prescribing information for 2,2′-dithio-bis-ethane sulfonate or journal discussion the same.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

Alternatively, 2,2′-dithio-bis-ethane sulfonate may be delivered orally using formulations that protect the compound from oxidation in acidic environments and allow intestinal absorption.'

“Non-smoker” means an individual who, at the time of the evaluation, is not a smoker. This includes individuals who have never smoked as well as individuals who in the past have smoked but have not used tobacco products within the past year. In one example, the term “non-smoker” means a human that has a smoking history of 15 pack-years or less, or who has not smoked for over 25 years. Appropriate categories can be selected with no more than routine experimentation by those of ordinary skill in the art. In certain embodiments the test subject is a non-smoker. A “never smoker” is an adult who has never smoked, or who has smoked less than 100 cigarettes in his or her lifetime.

The term “effective amount” as used herein refers to the amount of an agent needed to alleviate at least one or more symptom of the disease or disorder, and relates to a sufficient amount of pharmacological composition to provide the desired effect. The term “therapeutically effective amount” therefore refers to an amount of the agent that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

The dosage ranges for the administration of an agent according to the methods described herein depend upon, for example, the form of the agent, its potency, and the extent to which symptoms, markers, or indicators of a condition described herein are desired to be reduced, for example the percentage reduction desired for tumor growth. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of an agent described herein in, e.g. the treatment of a condition described herein, or to induce a response as described herein (e.g. lung cancer) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size and/or growth rate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of lung cancer in a mouse model. When using an experimental animal model, efficacy of treatment is evidenced when a statistically significant change in a marker is observed, e.g. tumor size and/or growth rate. A method of treating advanced and/or metastatic non-small cell lung cancer in female patients, the method comprising: administering to a human patient having non-small cell lung cancer who has received a second-line or a higher-line therapy a pharmaceutical composition of 2,2′-dithio-bis-ethane sulfonate, or a pharmaceutically-acceptable salt thereof and a second therapeutic agent.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

The term “treat” is used and includes both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

### EXAMPLES

The following examples are included for purposes of illustration and are not intended to limit the scope of the invention.

### Example 1

There are several pathways in NSCLC adenocarcinoma whose targets are often overexpressed in females, which disodium salt of 2,2′-dithio-bis-ethane sulfonate modulates. Therefore, disodium salt of 2,2′-dithio-bis-ethane sulfonate targets within the following key pathways: 1) kinases involved in key signaling pathways (ALK, ROS, MET, EGFR), 2) enzymes critical for DNA synthesis & repair (ERCC1, RNR1, RNR2), and 3) enzymes & proteins important in regulating cell redox status (Trx, Prx, Grx, PDI). The mutations and overexpression that are targeted and modulated by disodium salt of 2,2′-dithio-bis-ethane sulfonate are more likely in women with lung adenocarcinoma, especially non-smokers.

Results from clinical trials in disodium salt of 2,2′-dithio-bis-ethane sulfonate showed that female non-smokers had a survival increase from 13 months to 25 months, whereas results in all genders and smoking status groups saw marginally increased survival. Results from the trials exhibited an overall survival of 25.0 months, with a 2-year survival of 51.4%, in females with advanced adenocarcinoma of the lung receiving paclitaxel/cisplatin. The observed results were statistically significant (p-value=0.0477; HR=0.579) and were observed in a subgroup of 114 female patients upon retrospective analysis. Consistent statistically significant results were observed in a prior disodium salt of 2,2′-dithio-bis-ethane sulfonate double-blind, placebo-controlled trial in female adenocarcinoma patients conducted in Japan.

Disodium salt of 2,2′-dithio-bis-ethane sulfonate exhibits chemoprotective properties and reduces anemia, both of which disproportionately affect females. A Phase III Lung Trial also demonstrated important safety/toxicity profile advantages by protection against chemotherapy-induced kidney toxicity & reduced anemia. These data complement earlier clinical observations regarding disodium salt of 2,2′-dithio-bis-ethane sulfonate's ability to protect against neuropathy & other chemotherapy-induced toxicities.

### Example 2

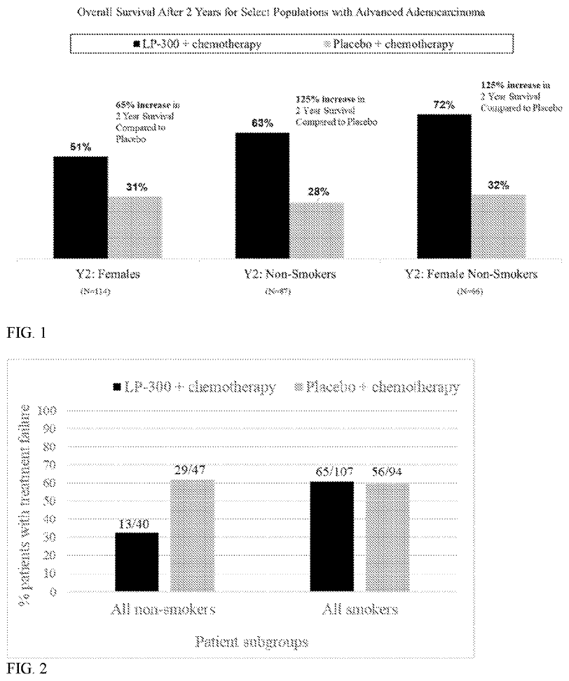

FIG. 1 shows retrospective subgroup analyses of NSCLC adenocarcinoma patients receiving Cisplatin/Paclitaxel from the phase III trial study ID DMS32212R (ClinicalTrials.gov Identifier: NCT00966914) showed a remarkable survival benefit in females, non-smokers and female non-smokers from the 2,2′-dithio-bis-ethane sulfonate treatment arm, as depicted by the overall survival improvements.

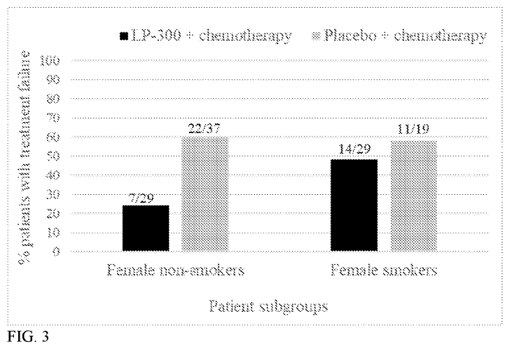

FIG. 2 shows the percentage of patients undergoing treatment failure was the least among non-smokers in the 2,2′-dithio-bis-ethane sulfonate treatment arm.

FIG. 3 shows the percentage of patients undergoing treatment failure was the least among females non-smokers in the 2,2′-dithio-bis-ethane sulfonate treatment arm.

Although the description referred to particular example embodiments, it will be clear to one of ordinary skill in the art that example embodiments in accordance with the invention may be practiced with variation of these specific details. Hence these example embodiments should not be construed as limited to the embodiments set forth herein.

1. A method of treating a female patient suffering from non-small cell lung cancer comprising the step of administering to the patient in need thereof a composition of 2,2′-dithio-bis-ethane sulfonate, or a pharmaceutically-acceptable salt thereof.

2. The method of claim 1, wherein the patient is a non-smoker.

3. The method of claim 2, wherein the non-small cell lung cancer is lung adenocarcinoma.

4. The method of claim 1, further comprising the additional step of co-administering to the patient in need thereof a second therapeutic agent useful in the treatment of non-small cell lung cancer.

5. The method of claim 4, wherein the second therapeutic agent is paclitaxel or cisplatin.

6. The method of claim 4, wherein the second therapeutic agent is selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, and cetuximab.

7. The method of claim 4, wherein the non-small cell lung cancer is EGFR mutant-positive non-small cell lung cancer.

8. The method of claim 1, wherein 10-40 grams per dose of the of 2,2′-dithio-bis-ethane sulfonate, or a pharmaceutically-acceptable salt is administered to the patient.

9. The method of claim 1, wherein the patient is a never smoker.

10. A method of treating advanced and/or metastatic non-small cell lung cancer in female patients, the method comprising: administering to a human patient having non-small cell lung cancer who has received a second-line or a higher-line therapy a pharmaceutical composition of 2,2′-dithio-bis-ethane sulfonate, or a pharmaceutically-acceptable salt thereof and a second therapeutic agent.

11. The method of claim 10, wherein non-small cell lung cancer is EGFR mutation negative non-small cell lung cancer.

12. The method of claim 10, wherein the second therapeutic agent is paclitaxel or cisplatin.

13. A method of treating a female, nonsmoking patient suffering from to non-small cell lung cancer comprising the step of
a. determining whether the patient is a non-smoker; and
b. administering to the non-smoker a composition of 2,2′-dithio-bis-ethane sulfonate, or a pharmaceutically-acceptable salt thereof.

14. The of method of claim 13, further comprising testing for EGFR mutants.

15. The method of claim 13, further comprising the additional step of co-administering to the patient in need thereof a second therapeutic agent useful in the treatment of non-small cell lung cancer.

16. The method of claim 15, wherein the second therapeutic is paclitaxel or cisplatin.

17. The method of claim 15, wherein the second therapeutic agent is selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, and cetuximab

18. The method of claim 16, wherein the effective amount of ranges of 2,2′-dithio-bis-ethane sulfonate, or a pharmaceutically-acceptable salt thereof ranges from 0.01-10 grams per dose.

19. The method of claim 13, further comprising determining the non-small cell lung cancer is ALK, ROS, MET, EGFR mutant-positive non-small cell lung cancer, ALK, ROS, MET, EGFR.

20. The method of claim 13, comprising determining the non-small cell lung cancer includes ALK and ROS1 gene fusions/rearrangements, EGFR gene mutations/deletions, and MET/HGFR gene amplifications.
